(19) United States
(12) Patent Application Publication
(10) Pub. No.: US 2016/0195514 A1
(43) Pub. Date: Jul. 7, 2016

(54) Methods for Genetically Diversified Stimulus-Response Based Gene Association Studies

(71) Applicant: COYNE IP HOLDINGS, LLC, Atlanta, GA (US)

(72) Inventors: Kevin P. Coyne, Atlanta, GA (US); Shawn T. Coyne, Smyrna, GA (US)

(21) Appl. No.: 14/915,891

(22) PCT Filed: Sep. 3, 2014
(86) PCT No.: PCT/US14/53819

§ 371 Date: Mar. 1, 2016

Related U.S. Application Data
(60) Provisional application No. 61/873,161, filed on Sep. 3, 2013.

(51) Int. Cl. G01N 33/50

(52) U.S. Cl. CPC G01N 33/5014

(57) ABSTRACT

Methods are provided for improving the impact of genetically diversified stimulus response gene association (GDSRGA) studies. The methods may involve developing subpopulations to be contrasted in GDSRGA studies by obtaining a biological sample from each donor of a population of donors; selecting a common cohort from the biological samples by obtaining at least a partial genomic sequence from each biological sample, aligning the sequences of the biological samples, and removing biological samples that cannot be sequenced accurately or fail to align; applying a test molecule or condition to the biological samples to induce phenotypically distinct responses among the members of the cohort; and segregating the biological samples into subpopulations based on the phenotypically distinct responses. These subpopulations may be used in GDSRGA studies.

### FIELD OF THE INVENTION

The present application relates to the field of gene association studies. Specifically, the application relates to methods involving the search for gene alleles associated with differential responses by test subjects in stimulus-response based gene association studies.

### BACKGROUND OF THE INVENTION

Since the dawn of civilization, philosophers and scientists have attempted to understand: (1) why human beings are as we are as a species (i.e. the commonality question), and (2) why human beings are different from each other (i.e. the diversity question). At a high level of abstraction, these questions can each be pursued in two contexts: endogenous (e.g. why are most adult human beings typically about five to six feet tall and what causes others to be unusually short or tall?), and exogenous, or responsive to a stimulus (e.g. why does a particular chemical cause one reaction in most human beings, and why does it cause another reaction in others?).

The discovery of the structure of DNA, and the subsequent decoding of the human genome created new opportunities to make progress on both the endogenous and stimulus-response versions of these questions. However, the pattern of progress has been different between the two due to important differences in available techniques. The present method addresses the relative weakness of techniques associated with the diversity question in a stimulus-response context.

In the endogenous context, genetic scientists have used the genome to explore both the endogenous-commonality question, which is not discussed here, and the endogenous-diversity question. One particular technique, gene association studies, has proven invaluable in discovering the role of genetic variation in driving phenotypic differences such as appearance, functionality, etc. of individuals. In these studies, subjects are separated into cohorts based on a phenotypic factor (such as height, eye color, etc.) that is common within a cohort, but different from cohort to cohort to a statistically meaningful degree. The genetic composition of the cohorts is then compared in order to isolate which genetic variations also statistically distinguish those same cohorts.

This approach has been developed quite extensively, including the techniques to analyze the patterns and degree of association of one or more genes. For example, scientists have developed multiple subsets of genetic information to examine various aggregations of single nucleotide polymorphisms, including but not limited to, whole genome, whole exome, specific regions of the genome or exome, or individually identified genes. They have utilized information that comes from the products of DNA, such as RNA through use of a transcriptome, rather than examining the DNA itself. They have looked to isolate single-locus gene effects, multi-loci effects, and main-versus purely-epistatic effects. They have examined both direct and indirect gene associations. And they have developed a variety of mathematical tools such as two dimensional matrices, heat maps, self-organizing maps, cluster analysis tools, etc.

In the stimulus-response context, gene association studies have been used primarily on the commonality question rather than the diversity question. For example, it is common practice to determine which gene (generally in the population) is associated with the response of a species to a chemical (stimulus) by applying the chemical to samples of tissue from multiple members of the species, then measuring which gene or genes shift their expression levels. However, the stimulus-response inputs for these techniques invariably rest on an unspoken premise—that the subjects to whom the stimulus is being applied (and from whom the response is made and subsequently measured) are scientifically equivalent (in the context of the experiment's goals) to each other. In other words, the results from various test subjects can be compared, because it is assumed that the results would have been the same had the match between any particular two test subjects and their respective stimuli been interchanged. Thus, for example, Waring and colleagues (2001) could compare the reactions of multiple genes to multiple chemicals using multiple rats, precisely because they assumed that each type of gene from every rat tested would respond the same as the same gene from any other rat tested.

In contrast, the use of gene association studies to attack the diversity question in stimulus-response situations (i.e. why does one human respond differently to the same stimulus as another human?) has proven more difficult. In commonality-centric stimulus-response work, the precision of response measurement can be relatively low (i.e. “did the subject respond or not?”), and small or even sometimes large differences between the responses of subjects ignored. In contrast, quite precise measurements of response may be necessary to distinguish the degrees of response that should define the various cohorts in a diversity study. Similarly, subtle or difficult-to-discern differences among test subjects may not matter in a commonality study (e.g. when findings are reported at the level of “most or all people”), but matter greatly in diversity studies. Finally, the far greater granularity in differences in responses that may matter in a diversity study dictate far greater diligence in discovering and eliminating any differences in extraneous stimuli (which merely represent “noise” in the signal-to-noise ratio in any measurement of responses).

Thus, the ability of scientists to precisely create test cohorts, and then precisely measure both the stimulus and response of those cohorts, have proven to be a barrier to reaping the full potential scientific benefit of genetic-diversity-stimulus-response-gene-association studies (referred to herein as GDSRGA studies). Further, limitations caused by these weaknesses in the integrity of the populations being studied, and weaknesses in the measurement of their respective responses, limit the types and precision of analyses that can be applied to such populations, as the precision and discrimination of any analysis is limited by the robustness of the underlying data itself.

The power of GDSRGA studies can be greatly enhanced by: (1) developing a new standardized panel population that eliminates many of the current limitations; (2) developing new protocols to control the experimental conditions that have previously caused weaknesses in the integrity of the sub-populations to be contrasted, as well as measurement of their respective responses; and (3) expanding the data sets and analytical comparisons that can be validly drawn from the response of the contrasted populations.

More powerful GDSRGA studies would be useful in a wide variety of fields. One exemplary field is the testing of pharmaceutical drugs for toxicity effects on humans, where a variety of problems and limitations currently exist. For example, despite the strenuous efforts of pharmaceutical companies to adequately test experimental pharmaceuticals, including the expenditure of millions of dollars and numerous years in pre-clinical testing such as in vitro and animal testing, a new pharmaceutical drug may cause adverse drug reactions in a small, but significant, portion of clinical trial participants or patients who take the drug after it has completed the regulatory approval process and been introduced into the marketplace. The resulting adverse drug reactions are often extremely costly, in both human and financial terms, for the individuals affected, the pharmaceutical companies, and society as a whole.

It is well established that genetic differences among human beings are one of three major causes of differences among persons in their reactions to drugs (the others being the age of the person and the environment to which the person has been exposed throughout his/her life), wherein the majority of persons may tolerate a particular drug with no adverse effect, while a small percentage of persons experience problems. Therefore, there is substantial interest in methods to determine the specific genetic causes of differences in drug response among humans.

Much of the work in this area has centered on GDSRGA studies, which attempt to determine the specific gene alleles that are statistically significantly more common in patients who suffered an adverse drug reaction than in other patients who took the same drug but did not experience an adverse drug reaction. GDSRGA studies have proven to be difficult, for at least the following reasons: (1) the data available for such studies has generally come from one-off clinical trials or actual post-regulatory-approval usage in patients, in which cases control conditions are not ideal for statistical analysis; (2) the obtainable data from these tests is constrained; (3) these constrained data sets in turn constrain the usable statistical analytical approaches and tests to relatively “low power” tests; and (4) the idiosyncratic nature of each of the clinical trials or patient experiences prevents the use of cross-drug data sets and new analytical approaches that could capitalize on cross-drug data patterns and learning.

These factors combine in negative ways such that GDSRGA studies have previously been characterized as being at the low end of the evidentiary hierarchy. What is needed, therefore, are methods that significantly improve the power of GDSRGA studies.

### BRIEF SUMMARY OF THE INVENTION

The methods and compositions described herein are directed toward improving the ability of GDSRGA studies to detect the causative gene alleles associated with differing reactions of various human beings, or specimens of animals, to certain stimuli, such as exposure to chemical or biological agents.

These methods and their application involve several inter-connected processes: (1) establishing a large scale, uniform cohort of cellular, tissue, organ, or organ system-type biological models (herein illustrated by pluripotent stem cell lines and their derivatives) that represent a highly controlled set of test subjects (referred to herein as a cohort of “donors”) who vary only in their genetic makeup, for use within a single study, and for use as identical cohorts when compared across many independent physical tests; (2) creating a fully sequenced and aligned set of genomes associated with those donors, and amending the cohort as dictated by that sequencing and alignment activity; (3) establishing the common set of experimental control procedures to be applied to all experiments using this cohort in order to extract more- and more-precisely-measured data that support highly sensitive scientific tests both within and across experiments; (4) applying previously unusable and/or novel analytical techniques to the data extracted from the one-time use of that cohort; and (5) applying previously unusable and/or novel analytical techniques enabled by the repeated use of that cohort across multiple experiments involving more than one compound and/or application to more than one cell type.

The methods can be applied across any GDSRGA study in which a researcher seeks to: observe or measure the response of “biological models” (defined as any aggregate or composition of individual cells from one donor held in vitro or in silico including, but not limited to, cells, tissues, organs, and organ systems) of a large number of subjects under specified common conditions; separate the subjects, based on that observation or measurement, into sub-populations of any size; and compare the genetic makeup of the subjects within some of those sub-populations to that of subjects in other of those subpopulations using any of the known methodologies, including but not limited to those described above in connection with the endogenous concept.

The methods can be used for, but are not limited to, examinations of the toxicity or efficacy of pharmaceutical drugs and vaccines; studies of the biological effects of other chemicals; studies of the susceptibility to, or propagation of, disease; studies of the impact of environmental conditions at certain exposures; and studies of nutrition. Further, the method can be applied not only to humans, but to all types of animals.

Given the many aspects of the method, and its broad applicability, a comprehensive discussion of every aspect in every application would be lengthy and could interfere with the ability to relate various aspects of the method to each other. Therefore, the remainder of this patent application is confined to the application of the method to an exemplary embodiment: using GDSRGA studies to analyze the genetic causes of toxicity effects of pharmaceutical drugs as measured through in vitro experiments. The applicability of the method to other uses can be readily inferred from this example.

### BRIEF SUMMARY OF THE FIGURES

Non-limiting embodiments of the methods of the invention are exemplified in the following figures. These figures illustrate three kinds of analyses supported by the methods described, as applied in the context of analyzing the genetic causes of toxicity effects of a pharmaceutical drug.

FIG. 1 is a bar graph showing a plot of toxicity of a test drug on a cohort of donors or subjects. The 500 donors are plotted in groups of 10 (i.e., one bar for every 10 donors) along the x axis in order of increasing toxicity severity score of the donor in response to the test drug. The level of toxicity severity score is plotted on the y axis.

FIG. 2 is a table showing the presence or absence of two alleles, A and B (each from a different gene) in each of 50 donors with high toxicity severity scores. A “1” in a column indicates the presence of the indicated allele type.

FIG. 3 is a bar graph, based on the data from the table in FIG. 2, showing the correlation between the presence of two alleles, A and B, and a donor's ranking among 50 donors with high toxicity severity scores. The 50 donors are plotted in groups of 10 (i.e., one bar of each color for every 10 donors) along the x axis based on their toxicity severity score (i.e., donors 1-10 being those with the highest toxicity severity scores among the 50 donors, and donors 41-50 being those with the lowest toxicity severity scores among the 50 donors). The y axis shows the percentage of cases in which the alleles are present. The presence of Allele A only is shown as the leftmost bar in each set of three bars (dark with white dots); the presence of Allele B only is shown as the middle bar in each set of three bars (solid); and the presence of both Allele A and Allele B is shown as the rightmost bar in each set of three bars (light with dark slanted lines).

### DETAILED DESCRIPTION OF THE INVENTION

The methods described herein are directed toward improving the ability of GDSRGA studies to detect the causative gene alleles associated with the differing reactions of various human beings, or specimens of animals, to certain stimuli, such as exposure to chemical or biological agents. The methods are illustrated herein through the embodiment of using GDSRGA studies to analyze the genetic causes of toxicity effects of pharmaceutical drugs as measured through in vitro experiments.

The methods may involve developing subpopulations to be contrasted in GDSRGA studies by obtaining a biological sample from each donor of a population of donors; creating a common cohort from those biological samples by obtaining at least a partial genomic sequence from each biological sample, aligning the sequences of the biological samples, and eliminating or removing from the cohort biological samples that behave inconsistently or disturb the alignment, such as the inability to be sequenced accurately or the failure to align; applying a test molecule or condition to the biological samples to induce phenotypically distinct responses among the members of the cohort; and segregating the biological samples into subpopulations based on the phenotypically distinct responses. These subpopulations may be used in GDSRGA studies.

The development of and use of these subpopulations in GDSRGA studies involve several inter-connected processes, which include, but are not limited to the following: (1) establishing a large scale, uniform cohort of cellular, tissue, organ, or organ system-type biological models that represent a highly controlled set of donors who vary only in their genetic makeup, for use within a single study, and for use as identical cohorts when compared across many independent physical tests; (2) creating a sequenced and aligned set of partial or complete genomes associated with those donors, and removing or elimination biological samples from the cohort as dictated by that sequencing and alignment activity; (3) establishing the common set of experimental control procedures to be applied to all experiments using this cohort in order to extract more- and more-precisely-measured data that support highly sensitive scientific tests both within and across experiments; (4) applying previously unusable and/or novel analytical techniques to the data extracted from the one-time use of that cohort; and (5) applying previously unusable and/or novel analytical techniques enabled by the repeated use of that cohort across multiple experiments involving more than one compound and/or application to more than one cell type. Each of these processes is described in detail below.

From a practical standpoint, until very recently, GDSRGA studies have been largely restricted to analyzing the results of real life events, such as the results of clinical trials of pharmaceuticals, or collecting and analyzing tissue samples from persons exposed to toxic environmental events. Limited work has been done using tissue samples for in vitro testing, but this has proven difficult, because of the limitations with respect to both quantity and timeframe associated with the use of primary tissues (i.e., the sample size is small, allowing for few replicates, and the cells die quickly), and because of suspicions about the “authenticity” of any responses from cancerous or engineered cells.

Thus, two characteristics of the “stimulus/response” side of GDSRGA studies have limited the analysis that can be conducted on the “causation” side (i.e., gene association). First, the data on response is usually “dirty”, in that the response can often be measured only crudely, and can often be the result of numerous stimuli other than just the one being studied. For example, responses collected in clinical trials is almost always subject to a variety of “contaminants” such as qualitative reporting of important responses (e.g. pain levels), inconsistencies in behavior or accuracy of reports by test subjects, and unreported contributing factors, such as exposures to stimuli other than that being studied (e.g. if a test subject was exposed to a toxic chemical, or to a contagious relative). This results in only qualitative experimental designs, and categorical (usually binary) assignment of test subjects into cases versus control status.

Second, the stimulus-response cycle cannot be repeated on the “same” experimental subjects, because (in real life experiments) the subject's own response to the first stimulus necessarily results in the subject being different in some way the second time.

Very recently, the discovery of large number of parallel pluripotent stem cell lines that can be cryogenically preserved without altering the cells' subsequent behavior (U.S. Pat. No. 7,569,385 to Haas or International Patent Application No. PCT/US2014/050762) provides the potential for creating the conditions that can reduce or eliminate these limitations. These stem cells provide an unlimited supply of cells that can be used on demand and allow experiments to be conducted sequentially. Thus, for the first time, researchers can develop and execute the experimental controls necessary to remove sources of stimulus other than the one to be studied, and can conduct repeat or follow-on experiments on the “same” (i.e., in this case, identical copies of) test subjects.

However, this ability to reproduce identical test subjects does not automatically confer the quality of repeatability. A number of additional innovations—which constitute the present invention—are necessary to achieve the experimental control that is essential to repeatability. These involve: (1) shifting the focus of experimental design away from any one experiment to those elements that must be controlled to be identical across all experiments in a meta-comparison set; (2) revising the rules that govern inclusion of subjects in the cohorts to be tested, often in ways that run counter to previously accepted norms for sample selection; and (3) narrowing the ranges of acceptable tolerances beyond those previously required in such stimulus-response experiments.

### DEFINITIONS

The terms “genetically diversified stimulus-response based gene association study”, “genetically diversified stimulus-response based gene association studies”, “GDSRGA study” or “GDSRGA studies” as used herein are defined as any study or studies intended to determine the genetic features, including but not limited to, single nucleotide polymorphisms, copy number variations, indels, and inversions that are statistically associated with a particular response by a biological test subject to an identified stimulus in contract to a different response by another test subject. A GDSRGA study may involve all of the nucleotides within the test subjects' genome, or any subset thereof, including but not limited to, whole genome, whole exome, specific regions of the genome or exome, or specifically identified subset of genes or non-coding locations. Further, GDSRGA studies specifically include both direct and indirect gene association methodologies such as linkage analysis or linkage disequilibrium analysis, and include single-locus and multi-loci studies. GDSRGA studies may utilize information about the composition of DNA directly, or utilize information that comes from the products of DNA, such as but not limited to RNA, through use of a transcriptome.

The terms “gene allele” or “gene alleles” as used herein refer to more than one variant of a particular gene to specific alleles of multiple different genes or to any combinations of gene alleles of different genes.

### Process 1: Establishment of Large Scale, Uniform Cohort Bank

A single large scale cohort with at least 30-40 donors, preferably 300-350 donors, or more preferably 500 or more donors of cellular, tissue, organ or organ-system-type biological models is obtained. The method is exemplified by using human pluripotent stem cell lines, and their derivative functional cells, such as cardiomyocytes. However, one of skill in the art will appreciate that any other suitable cell, tissue, organ, or organ type (including in silico applications) may be used in the described methods. The donors are specifically chosen to be phenotypically representative of the larger population of interest (e.g., the U.S. population, a particular tribe in Western Africa, or the world population), and the genetic inheritance of each donor is studied sufficiently to identify (and later mathematically correct for) so called “confounding effects” and population stratification issues.

In contrast to classical sample selection protocols, donors are obtained using methods that eliminate or minimize diversification along dimensions other than genetics. For example, the samples may be perinatal stem cells, in order to eliminate differences in response due to age differences among donors. Further, if perinatal stem cells are used, donors may be born in the same community and furthermore may be born at the same hospital (thereby increasing the likelihood that the mothers lived close to each other) and within a short period of time in order to minimize the differences in environmental conditions to which the mother has been exposed during pregnancy. For example, the donors may have been born within the same one two or three month time frame depending on sample size. As another example, the mothers of the donors may have lived in the same community and/or had the same occupation during pregnancy.

From this point on, all activities and data (including population stratification requirements, reactions to every dose of every drug, etc.) from every step and test are tracked on an individual donor basis, and the analysis is conducted at the level of an individual donor, rather than at an aggregated level.

The donor cell lines are individually validated by challenging them with, for example, pharmaceutical compounds of known and calibrated toxicity using highly controlled in vitro toxicity testing procedures well known to those in the field. These tests document the reaction of each individual donor to each control-drug under various doses. Any donor cell lines displaying responses that significantly interfere with achieving consistent results across multiple repetitions of experiments (such as inconsistent propensity to adhere to plates, and/or inconsistent and/or highly aberrant reactions) when using typical toxicity testing protocols are eliminated from the cohort. These donors are replaced with other donors who are phenotypically representative of the same segment of the population as the eliminated donors, and the entire population stratification process is recalibrated as necessary.

Process 2: Creation of Fully Sequenced and Aligned Set of Genomes with Amendment of Cohort Bank as Necessary

Next, the DNA of every donor is subjected to full or partial genome sequencing. All donor genomes in the cohort are then aligned on a global basis, for example by using a multiple sequence alignment software program such as, but not limited to BAli-Phy, Base-by-Base, ClustalW, DNA Baser Sequence Assembler, MAFFT, Phylo, PicXAA, and T-Coffee. Heuristic techniques may be used in the early stages of the alignment, but may not be used in the final round of sequencing. The final alignment must then be validated using a second global alignment optimization algorithm. Should any donor's DNA contain a unique feature that prevents it from being sequenced accurately (e.g. by requiring human judgment, the calling of base-pairs, more often than the third standard deviation of the number of cells for such sequences), or from being successfully aligned with the other genomes, the donor is eliminated from the cohort, and replaced in a procedure similar to that described above in Process 1 (including population re-stratification if necessary).

Each individual donor cell line within the cohort is then expanded according to the same protocol and using the identical growth factors and reagents across all donors. Expansion may be achieved using robotic cell culturing machines. The specific technique for expansion can be any one of many well-known to one of skill in the art. In certain embodiments, the expansion technique may be, for example, the one described in U.S. Pat. No. 7,569,385.

Importantly, records are kept of each passaging of the cells, and the expansion process for a particular cell line is stopped at a specific passage number, defined by two criteria: (1) enough cells have been generated from that line that all future uses of this cohort (including all differentiation into derivative cell types, as well as all experiments to be based on these cells or their derivative cells) will be based on cells of the same passage of that particular stem cell line; and (2) to the degree practically possible, the same passage stopping point is applied to all cell lines. This specification avoids phenomena known as “passage drift” and “genetic drift,” both of which can reduce comparability across donors and/or experiments.

Next, all cell lines are cryopreserved using a common protocol across all donors.

From this point on, membership in the cohort of donors is kept constant throughout all future experiments.

### Process 3: Establishment of Common Experimental Control Conditions Across Experiments

While experimental controls are standard industry practice for in vitro testing, the imposition of common controls both within an experiment and across experiments is novel in the field of GDSRGA studies, and such controls are necessary enablers of a number of advancements in GDSRGA study methodology and applications described later in this application.

Condition A

The researcher must establish, a priori, and strictly adhere to, all elements of the protocol that are common across all donors within an experiment. The objective is to remove all unintentional sources of variation other than the genetic differences among the donors. Therefore, common protocols should cover all factors that might impact results including, but not limited to: cryopreservation and thawing, cell count, reagents, incubation conditions, dosages, equipment itemization and/or specifications, and observation and measurement methodologies.

The creation of common experimental control conditions makes it possible to conduct the same experiment, or multiple experiments that vary only one variable, on biological models of the same donor multiple times to determine the variation of results that is inherent in the biological system. Later quantitative analysis of this variation, and incorporation of those findings into comparisons of results across donors, avoids much of the potential for false positives and false negatives that have been a feature of GDSRGA studies in the past.

Subsequently, when the variation among the replicates is narrow, statistical comparisons that have not been available to GDSRGA studies before, such as but not limited to, treating as significant only those inter-donor variations in reaction in which the minimum observation of any case member's reaction exceeds the maximum observation of any control population members' reactions, may increase the likelihood of finding a causative allele.

Condition B

The researcher must impose the same protocols and controls across multiple experiments. In order to compare results across experiments, or to combine the results of experiments into aggregated data sets for joint analyses, it is necessary to adopt the same objective to remove all unintentional sources of variation across experiments as were applied within experiments in the section above. Therefore, any new experiment must explicitly identify all elements that are to be deliberately changed from the preceding ones, and the preceding protocol must be varied only to the degree necessary to accommodate those specific changes. All other elements of the protocol are held constant to those in the preceding experiment.

In one embodiment of the value that accrues to this part of the method, those donors who exhibit a large end-point score to a drug in comparison with their own “average” end-point score when exposed to other reference drugs (such as in the same chemical class) are treated as the “cases” in subsequent analysis. This stands in contrast to the current practice of treating as cases those donors who exhibit a large end-point score (in absolute terms) for the particular drug under investigation compared only to the scores of other donors when exposed to the same drug. This new method of selecting cases is more consistent with a search for alleles that cause an individual to suffer a compound-specific severe reaction, rather than simply identifying alleles associated with sensitivity to an entire class of drugs. Such an analysis is key in the quest for personalized medicine.

Process 4: Application of Previously Unusable and/or Novel Analytical Techniques to Data Extracted from Cohort within GDSRGA Study

The application of common experimental controls in the present method, coupled with the highly comparable cohort of donor cell lines, greatly narrows the margin of error associated with any type of measurement of the behavior of the cells. This enables the method to make novel use of three types of allele search strategies and analysis that are new to GDSRGA studies:

Strategy A—Deploy gene allele search strategies that rely on more precise measurements of a commonly used end point to create novel groupings of test subjects for genomic comparison.

The move from simplistic broad divisions (e.g., binary case/control divisions) to “continuous quantitative measurement” opens the possibility of novel groupings that include, but are not limited to: (1) gene search strategies that compare and contrast only the genomes of sub-segments of the population with the greatest degree of difference in the measured behavior; and (2) gene search strategies that segment the cohort based on inflection points in the degree of reaction, rather than using binning strategies (such as deciles, quintiles, etc.).

This procedure maximizes the difference in level of reaction between the control population and the case population, thereby maximizing the likelihood of genetic differences. The comparison is made between these novel sub-populations using familiar GDSRGA techniques to identify causative alleles.

In a related subsequent embodiment, if a gene allele has been identified through the above analysis as being associated with the degree of reaction to the drug, each of the genomes of all donors in the entire cohort are examined for the presence of the suspect allele, beginning from the single most severely affected case, and proceeding sequentially towards the least affected case. The data from those donors with the identified allele who also suffered source reactions is then used to recalculate the size of the case population and compute a new power and confidence level.

In a third related and subsequent embodiment, the ordered list of donors and their respective quantified reactions are sequentially examined for any significant changes in genetic patterns at particular points in the distribution. Here, a map of the presence (or absence) in each test subject of the allele identified above is generated, compared to the quantified levels of reactions, and the two are jointly analyzed to determine whether there are discernible points where attention should be focused to determine whether any of several significant changes in the presence of gene alleles has occurred. For example, one change may be that all donors with higher reactions have the suspect allele, whereas those with reactions below that point do not have the suspect allele. A second change may the new appearance of a second gene allele (either of the same gene, or of a different gene) common to the next group of donors, but absent in either the first group or groups with still lower reactions.

In a fourth embodiment, the graph arranging donors in ascending order of impact may reveal particular inflection points, where the level of reaction of a donor rises disproportionately compared to its next lower neighbor than had been the case when comparing earlier neighbors in the cohort (defined as donors for whom the percentage difference in reaction score compared to the score of the previous donor significantly exceeds the comparable measure associated with other donors in the vicinity on the ordered list). This point can then be used as the demarcation point for comparing the genomes of the subpopulations to the left and right of that point.

Strategy B—Deploy gene allele search strategies that rely on new end points that were previously considered unmeasurable per se, or where differences in reaction among participants were previously considered too subtle to attempt measurement.

Examples include, but are not limited to: (1) collecting parameters at times other than the terminal end point (such as the degree of effect at a given point in time during the experiment) rather than only taking measurements after the experiment is completed, as is the typical protocol today; or (2) collecting new vectors of information (such as the dosage that achieves a certain threshold of impact, or functional measurements within the cell such as mitochondrial activity or ion channel activity) that can only be captured when the experiment can be replicated (e.g., with different concentrations) on the same donor under the same experimental conditions.

In one embodiment of this type of search strategy, the typical comparison of cell death rates among donors exposed to a single specified dose of a compound under investigation is eschewed in favor of focusing on the dosage or concentration level required to produce a threshold level of effect (e.g., the dosage required to cause cell death in 20 percent or more of the cells challenged). In another embodiment, the focus shifts to the time required for a threshold effect (e.g., a cell death rate of 20 percent) to occur.

Process 5: Application of Previously Unusable and/or Novel Analytical Techniques Enabled by Repeated Use of Cohort Across Multiple Experiments

Because the use of a common pool of cells, common donor cohort, and common protocols across experiments eliminates many sources of unintended variation between experiments, it is now possible to use results from one experiment to inform the conduct of the search for causative alleles in other experiments, and both physical and genomic results of experiments can be combined to form insights not previously obtainable. Such techniques and their associated lessons (which can be combined with the techniques described in the previous section) include, but are not limited to:

Technique A—Deploy gene allele search strategies that rely on forming case and control populations based on a test subject's “simultaneous” reaction along multiple parameters that cannot be measured in the same physical experiment.

Many types of physical tests (such as certain biological marker tests) cannot be deployed simultaneously, as the very conducting of one test interferes with the data generated by the other. In such cases, researchers have been limited to deploying only one of the fratricidal tests. Moreover, because the supply of experimental subjects was exhausted by the first test, or the subjects themselves were altered by that first test such that they can no longer be considered “equivalent” to the first set of subjects, researchers have had no ability to cross-compare the results of multiple tests on the same individual donor. With the present method's capability to create an unlimited number of equivalent replicates for each donor, the results of any number of otherwise fratricidal tests can be cross-compared to form novel case and control populations.

For example, all variants of a Venn diagram analysis of the parameters of interest can be included, such as: (1) selecting as the case population those donors who displayed a reaction within a certain range on one parameter while also displaying a reaction within a (different) certain range on another parameter; (2) selecting as the case population those members who displayed either a response within a certain range on one parameter or a response within a certain range on a second parameter; or (3) selecting as cases those members displaying other multi-parameter behavior inclusion and exclusion criteria, such as displaying response A but not response B, etc.

Technique B—Conduct cross-experiment comparisons and contrasts.

A variety of cross-comparisons are useful in the search for causative gene alleles, as well as for developing a greater understanding of the functioning of the genes themselves.

In one embodiment, multiple new case-versus-control populations are developed from a given set of experiments, by selecting as cases only those individuals who had (either absolutely or relatively) higher end-point scores when challenged by one compound than when challenged by another compound. For example, it is possible to ask (for the first time) whether a given statin adversely affects any specific individuals significantly more or less than another, previously analyzed statin, and if so, whether the causative alleles might be different than those previously identified from a GDSRGA study using case-control populations drawn from the previous drug.

Another embodiment involves comparing individual donor results across different functional cell types when challenged by the same compound (e.g., comparing the results when using cardiomyocytes versus hepatocytes from the same donor). Should there be a significant difference in (either absolute or relative) reaction by one cell type versus (an) other cell type(s), and should that difference hold true across a number of donors' cells, then any gene allele(s) identified though a GDSRGA study based on the higher reacting donors serving as cases would be a gene allele associated with both the compound and the specific functional cell type. Therefore, it can be hypothesized that the gene itself is one that directly impacts the function of that particular tissue. This can aid in identifying the function of previously unexplored genes.

Technique C—Apply learning and successful search strategies from one experiment to another.

Because biological processes and reactions can be caused by the interactions among multiple genes and among specific alleles of multiple genes, the number of possible genetic causes for a single effect may exceed the number that can be searched comprehensively, even with the most powerful computers in existence. This is particularly true for multigene causes and epistatic effects where genes can accelerate, retard, or alter the effects of other genes. Therefore, today scientists are forced to revert to heuristic techniques in their search for causative alleles.

A principle of such heuristics is that the closer the new situation being investigated matches a past (better understood) situation, the more likely that the solution in the past will approximate the present solution. However, in the past, so many parameters varied across every experiment that it was difficult to tell which prior situations were truly closer matches to the one being investigated now. Thus, lesson sharing strategies contain a large random element, and constitute little more than informed guesses. This creates significant potential for underlying causal alleles to remain undetected, despite substantial search effort.

With the present method's intra-experimental and cross-experimental controls, it is now possible to be systematic in assessing such closeness across experiments, thus improving the heuristics through trend spotting, linear and non-linear extrapolations of patterns, etc.

In one embodiment, the search space is limited and available search resources are used more efficiently (including the search for epistatic effects) by focusing on the gene regions previously identified as being associated with toxicity when other members of the same drug class were analyzed. Further, the findings from these earlier studies are used to develop specific hypotheses to test.

Beyond this, searches can become systematic without being forced to be comprehensive. For example, in another embodiment, data collected from a planned succession of similar experiments that deliberately and systematically vary individual design parameters are compared to see which ones do and do not cause escalating effects; then, the gene allele search is only conducted once the experimental outcomes have been optimized for discrimination.

Technique D—Synthesize individual experiment findings into “class” findings.

Until now, researchers needed to exercise significant restraint in hypothesizing commonalities about the impacts of any two or more stimuli (delivered independently). A researcher could comment on statistical measures only. For example, a researcher could say, “Compound A caused 14 test subjects to react, while Compound B caused 10 to react,” but comparisons could not be made at the individual donor level. The present method enables a greater level of specificity, and hence greater insight. For example, continuing the present example, a researcher could now say, “Of the 14 donors that Compound A caused to react, Compound B caused no reaction in 12 of them. However, in addition to causing 2 of the 14 to react, Compound B caused 8 donors who had had no reaction to Compound A to react.”

In one embodiment, individual donor level results of multiple experiments conducted within related sets (such as several compounds within the same chemical class) are compared to find commonalities and infer general patterns of impact. These range from findings at the reaction level to statements about the underlying causative alleles. For example, it is possible to find whether individuals with certain alleles have adverse reactions to all drugs within a class, or whether there is value to matching a specific individual with a specific drug within a class (i.e., personalized medicine).

It should be understood that the foregoing relates to certain embodiments of the invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein may suggest themselves to those skilled in the art without departing from the spirit of the present invention and/or the scope the appended claims.

### EXAMPLES

The present invention may be better understood by reference to the following non-limiting examples.

In certain embodiments, the methods described herein enable those who are developing new pharmaceutical drugs to implement a comprehensive program designed to more precisely understand the various toxicity effects of a candidate drug under development, so that it is possible to pursue one of four possible courses of action based on the results of the testing program: (1) abandon the compound; (2) refocus research efforts on a related compound that demonstrates equal or nearly equal efficacy while demonstrating lower toxicity; (3) alter the metabolized chemistry of the compound itself (for example, by developing a buffer for use in conjunction with the compound, to maintain its efficacy while reducing its toxicity); or (4) develop a genetic pre-screen to prevent those individuals who might be susceptible to a toxic reaction from using the drug. Importantly, depending on the specific circumstances involved, any one of these four courses may be superior to the only course of action that was previously available, which was to simply naively continue developing the drug until discovering that it fails clinical trials.

Three examples are presented here, each illuminating separate claims below.

### Example 1

### Establishing the “Platform” for Multiple Enhanced Gene Association Studies

This example discloses the establishment of the platform for multiple enhanced gene association studies—i.e., a large, highly consistent quantity of cells for a large cohort of highly consistent cell lines, the associated genetic data, and common underlying experimental controls. In this embodiment, the purpose is to test multiple candidate pharmaceutical compounds to estimate the portion of people in the U.S. who would be adversely affected by a given compound, by conducting in vitro testing using a particular stem cell obtained from neonates, or newborn human infants (as described, for example, in U.S. Pat. No. 7,569,385), with pre-established endpoints as the indicator of adverse effects. Further, it is assumed that the chosen end point is, “percent of cells that fail to survive for 10 days under incubator conditions after administration of the compound, as judged by the MTT staining test”.

The first step is to design an appropriate size and composition of a cohort of stem cell lines to be created. A final cohort sample size of 500 is selected, after: (1) determining from well-known statistical methods that a sample size of 500 will create a 99 percent probability that at least one member of the cohort will exhibit an adverse reaction if the true incidence in the U.S. population would be 1 percent or greater; and (2) assessing other critical issues including cost, access to sources of cell donors, sample sizes required for certain statistical tests, number of subdivisions of the sample that are to be separately examined statistically, etc.

The next step is to partition the total cohort sample size into target sizes for specific relevant subpopulations, in order to correct for certain confounding factors in the conversion of sample findings to population estimates. Prior art has established that there are only two known phenotypically-discernible factors in newborn infants that affect an individual's propensity to experience adverse drug reactions: race and gender. In order to facilitate and strengthen later statistical analysis, it is determined that the minimum size of any gender-race sub-cohort will be 30. From the U.S. Census, it is known that Caucasians make up 72 percent of the population, Blacks 13 percent, and Asians 5 percent (with the remaining 10 percent being of mixed race or belonging to one of several very-low-incidence races), and that males and females each make up roughly 50 percent of the U.S. population. Based on these percentages, a decision is made to allocate the 500 available sample “slots” into stratified samples as follows: Caucasian Females, 187 (or 37 percent); Caucasian Males, 187 (or 37 percent); Black Females, 33 (or 7 percent), Black Males, 33 (or 7 percent); Asian Females, 30 (or 6 percent); and Asian Males, 30 (or 6 percent). Standard statistical techniques for stratified samples (including using overall averages for the un-sampled very-low-incidence races) will be utilized to scale up any findings to the U.S. population.

Past experience with establishing cell lines from this particular source stem cell shows that the cells of 10 to 15 percent of donors will likely fail the genome alignment step that will be applied later. Therefore, the specified numbers of samples of source stem cells is increased by 25 percent. Thus, the actual number of samples to be collected are set as follows: Caucasian Females 234; Caucasian Males, 234; Black Females, 42; Black Males, 42; Asian Females, 38; and Asian Males, 38.

In order to ensure maximum consistency across the resulting total of 628 samples, the protocols that are typically used to create comparable cell lines are revised—for each step in the process, from collecting source tissues, to isolating the cells of interest, to expanding the stem cells—to be much stricter than those that would normally be used to simply create 628 cell lines. For example, it is specified that all donors be sourced at the same hospital within a three month period of time, and isolation and expansion steps are physically undertaken via a robotic fluid-handling and incubation system.

At this point in the example, an issue arises that could reduce the level of standardization across the 628 samples. Specifically, in the past, stem cell researchers have had concerns about the impact of batch-to-batch inconsistency of reagents. The traditional solution has been to ensure that any reagents used originate from a single batch at the manufacturer. However, in this case, it is not an option to specify that all 628 donors' cells be cultured using reagent from a single batch, because the reagent has an expiration time of four months, while in this instance the collection and processing of donors' cells must be spread out over eight months. To improve the consistency of the reagent across donor samples that must be collected and processed at different times, it is specified that the collection be divided into two periods. It is then specified that a large batch of reagent (capable of processing the cells of 314 donors, or half of the total donors) is to be created at the laboratory at the beginning of each of the two time periods by mixing smaller quantities of reagent from at least four different source batches obtained at that time from the same manufacturer. Thus, each of the two resulting large batches consists of the same “average” blend of four or more smaller batches, and therefore its composition is likely to be close to the mean composition of all batches. This reduces the potential for cell expansion in a subset of donors being nonstandard as a result of the composition of any single batch of the manufacturer's reagent deviating from the mean of the manufacturer's specification.

After designing these highly standardized protocols, particular attention is devoted to ensuring that the protocols are strictly adhered to throughout the execution of the process.

Once the cells from any one donor have been isolated and initially expanded, subsets of those cells are exposed to five concentrations of a standard compound (in this case ATRA), and an MTT cytotoxicity test is performed according to standard protocols. Any donor whose cells exhibit either extreme sensitivity (defined as more than 80 percent dying when exposed to the lowest concentration), extreme insensitivity (defined as fewer than 20 percent dying when exposed to the highest concentration), or inadequate concentration-responsiveness (defined as less than 20 percent variation between cell death percentages between the lowest and highest concentrations) is rejected at this point. Further, any donors whose cells behave inconsistently between replicates on any dimension that could interfere with comparability across experiments (such as failing to adhere to the plate in some, but not all, replicates) are also rejected at this point. In this example, three donors, all from the Caucasian Male group, are rejected.

Each time a remaining donor sample has been successfully processed to create its first batch of stem cells, but before those cells are cryopreserved, a small quantity of cells is separated and prepared for genetic sequencing. The full genome of that donor is then sequenced according to the sequencer manufacturer's specified protocol, adjusting the manufacturer's specs as necessary to ensure maximum accuracy. Despite the redundancy built into a single run, and the resulting accuracy claim alleged by the sequencer manufacturer, the read accuracy is checked by comparing the results of two independent readings of the same donor's sample, and the sequence is accepted only when there is a greater than 99.9 percent confluence between the two analyses. As a result of this process, 35 additional donors, spread among the six sub-populations, are rejected.

Once the donors have been sequenced and the 38 donors who failed the quality standards (i.e., three donors based on the first screen, then 35 donors based on the second screen) have been rejected, the required number for each sub-population (e.g., 187 for Caucasian Females) are randomly selected, and the process of aligning the genomes begins.

The global alignment process begins with simpler alignment models, but the penultimate alignment is an optimization based on a deterministic version of iterative dynamic programming. The contribution of each of the individual 500 donors' genomes to the aggregate alignment score is then calculated, as well as the “shadow” contribution of each of the 90 remaining “spare” donors (i.e., the original 128 “spare” donors, less the 3 who were rejected for concentration sensitivity issues, less the 35 who were rejected for initial gene sequencing issues). Statistics show that three of the 500 genomes may be extreme outliers in their genetic composition. Therefore, the alignment can be improved (without sacrificing any integrity regarding the randomness associated with the target 500 sample size against the larger population) by substituting three of these remaining donors for three of the original 500 in the alignment, ensuring that, in every case, the trade-out is made from within the same race-gender subpopulation. The optimization step is then repeated to ensure that the alignment is truly optimized for the new cohort of donors.

At this point, an additional set of protocols are employed in the expansion, differentiation and storage of the cell lines to create strict standardization across the repetitions of the process that underlie the ability to conduct many cross-comparable experiments on a single donor, while also continuing to provide standardization across the 500 donors in the cohort.

It is determined that this particular cohort will be designed to support up to 1,000 separate “experiments.” Each of these experiments will consist of applying, in a separate vial for each of the 500 sample members of the cohort, one compound at one concentration to a collection of 1,000 cells from that one member. Thus, for each of the 500 members of the cohort, a total of 1,000,000 cells must be possessed at the test point, and these must be aliquoted into 1,000 separate vials containing 1,000 cells each.

In order to ensure that the cells in each of the 1,000 vials are thoroughly consistent vial-to-vial, it is determined that, for each of the 500 source donors, a single cell will be isolated, then cloned until there are 1,000,000 cells, rather than begin with all of the cells isolated from the tissue sample. Further, while achieving 1,000,000 cells from a single cell clone theoretically requires 21 population doublings, past experience with isolating and expanding these types of cells shows that there is actually a distribution of results among donors with respect to the percentage increase in cell count that results from a single “doubling,” ranging from 1.91 to 1.96. Therefore, at least 22 doublings will be needed, and in some cases 23 doublings will be needed. Because of the importance of using the same passage across all donors, it is determined that all donors should be expanded to 23 doublings, even though, for most donors, there will be more than the 1,000,000 required cells available after 22 doublings.

As with earlier steps, each of the steps required for the expansion, differentiation and storage of the cells (such as aliquoting the cells into lots of 1,000 cells per vial) are physically undertaken, to the maximum degree possible, via robotic systems.

### Example 2

### Conducting Enhanced Gene Association Analysis within a Single Experiment

In this example, in vitro toxicity tests, at various concentrations of a particular compound, are conducted on the 500 members of the highly standardized cohort. One of the data outputs from that testing is an indicator of toxicity for which a “normal” score is below 2.0, and a score of 7.0 or above is considered “significantly elevated toxicity susceptibility.”

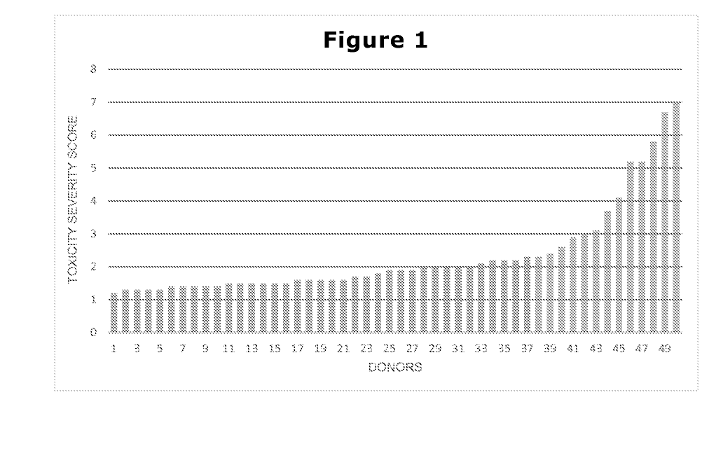

Results from the test are shown at the end of this patent application as FIG. 1, in which the donors are arranged from lowest score to highest, with one bar representing 10 donors. Numerically, the scores for 270 donors are below 2.0, while the scores for 10 donors are 7.0 or above. The median donor scores 1.9; the lower quartile scores 1.5; and the upper quartile scores 2.3.

In this instance, standard attempts at gene association fail to produce any identifiable allele association with the toxic effect. Not enough donors have reached the “significantly elevated toxic susceptibility” cutoff point (7.0 or above) to enable a statistically confident comparison against the others—i.e., although 10 donors have reached that level, a minimum of 14 would be required to achieve greater than 80 percent confidence. In addition, comparisons of the 230 “above the norm” donors to the 270 “normal” donors have produced no statistically adequate differentiation in the rates of presence of any particular allele. Finally, a comparison of quartiles shows only that there are weak correlations involving two alleles when the highest-reacting quartile is compared to the second-highest-reacting quartile.

At this point in the example, a set of novel analyses employing portions of the method described herein are undertaken.

First, the minimum cutoff number of 14 described in the preceding paragraph is used to select those 14 donors with the highest reaction scores to establish a “case” group, ignoring the arbitrariness of the 7.0 threshold. Next, the 200 donors with the lowest reaction scores are chosen to establish an artificial “control” group, as 14 cases compared to a control group of 200 provides statistical confidence of 80 percent that any alleles identified are truly different between the two groups. This analysis identifies two alleles, A and B, each located on a different gene. Even with no further analysis, these highly useful findings will be reported to the pharmaceutical company that sponsored this research.

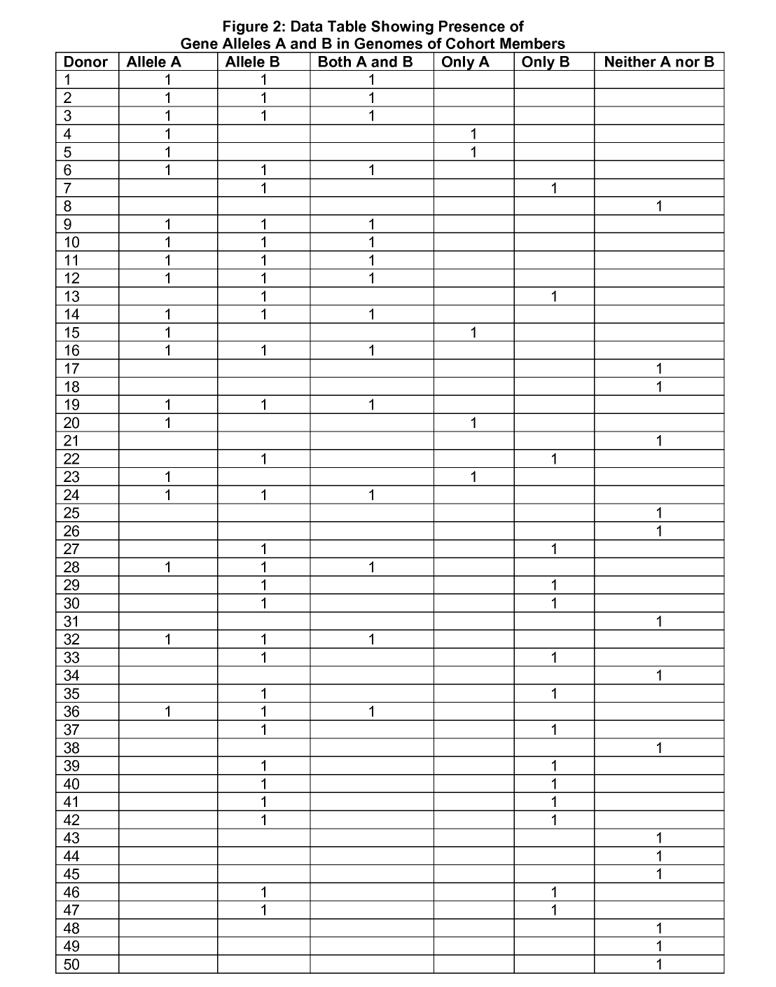

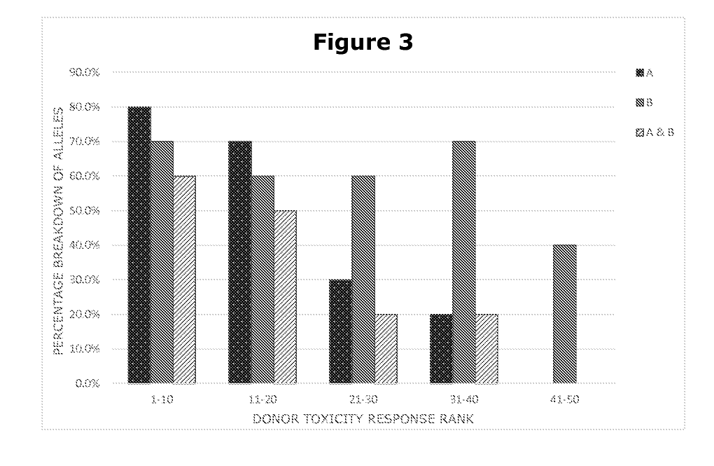

The next step is to examine the genomes of each member of certain sub-cohorts within the entire cohort, such as the 50 donors with the single highest reaction scores, to look for the presence of each of the two alleles, A and B, or both alleles. The results of this exemplary sub-cohort are shown in FIGS. 2 and 3. The figures show that there is a strong correlation between the presence of Allele A and a donor's ranking within the cohort. Specifically, 80 percent of the ten highest-scoring donors have the presence of Allele A, while 70 percent of the next ten have the presence of Allele A, then 30 percent of the next ten, then 20 percent of the next ten, then zero percent of the next ten. Therefore, a probable causative pattern is quickly identified that can then be subjected to more rigorous statistical testing.

Meanwhile, Allele B shows more of a constant presence, being present in 70 percent of the ten highest-scoring donors, then 60 percent of the next ten, then 60 percent of the next ten, then 70 percent of the next ten, then 40 percent of the next ten. This information leads to a conclusion that understanding Allele B's impact requires continuing further down the rank-ordered list of donors. Doing so shows that Allele B is often present throughout the highest-scoring quartile of donors, but is actually rare below that level. Again, a new hypothesis has emerges that can then be rigorously tested.

Beyond the individual alleles' scores, the figures also show that both Alleles A and B are highly prevalent among the ten highest-scoring donors (60 percent of cases); then the incidence of both Alleles A and B being present declines with each of the next groups of ten—to 50 percent among the next ten, then 20 percent among the next ten, then 20 percent among the next ten, then zero percent among the next ten. Thus, it is reasonable to hypothesize that there may be strong epistatic effects of the two gene alleles when they appear together.

Once the statistical analysis of Alleles A and B is completed, a third search strategy is used to look for alleles that have weaker, but still significant, effects. A closer examination of the graph in FIG. 1 reveals that there appear to be several inflection points where the toxicity score appear to jump-shift upward. Therefore, beginning with the donor who has the lowest score and proceeding upwards, the percentage difference in each donor's toxicity score versus the score of the previous donor is calculated. The arithmetic confirms that there are indeed points where the percentage difference for a given donor is statistically significant from that of the other donors around that donor. The relevant regions of the genomes of the ten donors who follow that inflection point are then compared to the ten that precede that inflection point, to see if there appears to be a single allele change between the two groups. Again, this process produces candidates for further investigation.

### Example 3

### Conducting Enhanced Gene Association Analysis by Comparing Results Across Experiments

In this example, in addition to testing the compound of interest, the same protocol is employed to conduct toxicity tests of three other compounds that are already on the market and have the same therapeutic purpose. Results from all four compounds are tracked on an individual donor basis.

One key analysis that is conducted is to compare the toxicity test score (as described above in Example 2) for each individual donor under challenge by the compound of interest to the toxicity test score of that same individual donor when challenged by each of the other three compounds. The measure employed is to divide the score generated by the compound of interest by the score generated by each of the other compounds. Donors for whom the resulting measure is above 2.0 (meaning that the toxicity reaction to the compound of interest was twice as strong or greater compared to the toxicity reaction of one of the other compounds) are identified as “pre-cases.” Next, the donors in the subpopulation of that pre-case group who also exhibit absolute toxicity scores of 4.0 or higher (i.e., twice the “normal” score of 2.0 on the scale described above in Example 2) are designated as the “case” population for use in a case/control gene association analysis. Thus, cases consist of only those who have both a high absolute score as well as a high relative score. Further analysis identifies an Allele X that is associated with the unique toxicity properties of this particular compound.

While the invention has been described and illustrated with reference to certain embodiments thereof, those skilled in the art will appreciate that various changes, modifications and substitutions can be made therein without departing from the spirit and scope of the invention. All patents, published patent applications, and other non-patent references referred to herein are incorporated by reference in their entireties.

What is claimed is:

1. A method of developing subpopulations to be compared in genetically diversified stimulus-response gene association studies comprising
a. obtaining a biological sample from each donor of a population of donors;
b. selecting a common cohort from the biological samples by obtaining at least a partial genomic sequence from each biological sample, aligning the sequences of the biological samples, and eliminating from the cohort biological samples that cannot be sequenced accurately or fail to align;
c. applying molecules or conditions to the biological samples to induce phenotypically distinct responses among the members of the cohort; and
d. segregating the biological samples into subpopulations based on the phenotypically distinct responses
wherein the subpopulations are contrasted in genetically diversified stimulus-response gene association studies.

2. The method of claim 1, wherein the genetically diversified stimulus-response gene association studies are to be performed on animals, mammals, or humans.

3. The method of claim 1, wherein the biological sample is an organ system, organ, tissue, cell, stem cell, multipotent stem cell or derivative thereof, or pluripotent stem cell or derivative thereof.

4. The method of claim 3 wherein the biological sample is contained either in vitro or in silico.

5. The method of claim 1, further comprising creating a plurality of identical copies of the cohort of biological samples and separately storing those copies for use in future experiments involving this cohort.

6. The method of claim 5, wherein the copies are cryogenically preserved.

7. The method of claim 1, wherein the test molecules or conditions consist of a small molecule pharmaceutical drug, biologic agent, vaccine, industrial chemical, pathogen, toxin, or environmental condition.

8. The method of claim 1, wherein the phenotypically distinct populations are defined by a quantified measurement of the amount of the test molecule or degree of the environmental condition necessary to cause a specified degree of response.

9. The method of claim 1, wherein the phenotypically distinct populations are defined by the time interval between exposure to the test molecule or environmental condition and the time at which a specified degree of response occurs.

10. The method of claim 1, wherein the phenotypically distinct populations are defined based on the test subjects' responses across multiple parameters measured in the same experiment.

11. The method of claim 1, wherein the phenotypically distinct populations are based on test subjects' responses based on one or more parameters measured in two or more independent experiments.

12. A method of conducting genetically diversified stimulus-response gene association studies comprising
a. obtaining a biological sample from each donor of a population of donors;
b. selecting a common cohort from the biological samples by obtaining at least a partial genomic sequence from each biological sample, aligning the sequences of the biological samples, and eliminating from the cohort biological samples that cannot be sequenced accurately or fail to align;
c. applying a test molecule or condition to the biological samples to induce phenotypically distinct responses among the members of the cohort;
d. segregating the biological samples into subpopulations based on the phenotypically distinct responses, and
e. contrasting responses to the test molecule or condition between subpopulations, wherein a positive response indicates an efficacious stimulus and a negative response indicates an adverse stimulus,
wherein the donors are obtained using methods that eliminate or minimize diversification other than genetics.

13. The method of claim 12 wherein the donors are within one year of a predetermined age when the samples are collected.

14. The method of claim 12 wherein the donors are neonates.

15. The method of claim 14, wherein the biological samples are perinatal stem cells.

16. The method of claim 14 wherein the donors have been exposed to similar environmental conditions during gestation.

17. The method of claim 16 wherein the donors were born in the same community, the mothers of the donors lived in the same community during pregnancy, the mothers of the donors had the same occupation during pregnancy or the donors were born within a predetermined time frame.

18. A method of segregating a cohort of test subjects into two or more phenotypically distinct populations for conducting genetically diversified stimulus-response gene association studies comprising
a. obtaining a biological sample from each donor of a population of donors;
b. selecting a common cohort of test subjects from the biological samples by obtaining at least a partial genomic sequence from each biological sample, aligning the sequences of the biological samples, and eliminating from the cohort biological samples that cannot be sequenced accurately or fail to align;
c. applying test molecules or conditions to the biological samples of the cohort of test subjects to induce phenotypically distinct responses among the members of the cohort; and
d. segregating the biological samples into two or more subpopulations based on the phenotypically distinct responses
wherein the two or more populations are defined by predetermined ranges of a quantifiable measure of the test subjects' response to the test molecules or conditions.

19. The method of claim 18, wherein only a subset of the cohort on which testing has been performed is included in the genetically diversified stimulus-response gene association studies,
wherein the subset comprises test subjects whose response is highest for inclusion in a case subpopulation, and test subjects whose score is lowest for inclusion in a control subpopulation.

20. The method of claim 18, wherein only a subset of the cohort on which testing has been performed is included in the genetically diversified stimulus-response gene association studies,
wherein the subset comprises test subjects whose response is lowest for inclusion in the case subpopulation, and test subjects whose score is highest for inclusion in the control subpopulation.

21. The method of claim 18, wherein the test subjects are separated into multiple subpopulations based on inflection points in the level of response by a test subject when compared to the responses of test subjects receiving the next higher and next lower scores, and wherein all donors are placed into subpopulations.

22. The method of claim 18, wherein the test subjects to be separated comprise only a subset of the test subjects in the cohort.

23. The method of claim 18, wherein the phenotypically distinct populations are defined by a quantified measurement of the degree of stimulus necessary to cause a predetermined degree of response.

24. The method of claim 18, wherein the phenotypically distinct populations are defined by the time interval between exposure to a stimulus and the time at which a predetermined degree of response occurs.

25. The method of claim 18, wherein the phenotypically distinct populations are defined based on the test subjects' responses across multiple parameters measured in the same experiment.

26. The method of claim 18, wherein the phenotypically distinct populations are based on test subjects' responses based on one or more parameters measured in two or more independent experiments.

27. The method of claim 18, wherein the separate and distinct ranges are defined using an algorithm that mathematically compares a test subject's response to a stimulus to the test subject's response to another stimulus, regardless of whether the stimuli were generated in the same experiment or in different experiments.

28. The method of claim 18, wherein the separate and distinct ranges are defined using an algorithm that mathematically compares a test subject's response to a stimulus to a different test subject's response to an identical stimulus, regardless of whether the stimuli were generated in the same experiment or in different experiments.

29. A method for identifying which cell, tissue, organ, or organ type is affected by a particular gene allele, comprising
a. obtaining a biological sample from each donor of a population of donors;
b. selecting a common cohort from the biological samples by obtaining at least a partial genomic sequence from each biological sample, aligning the sequences of the biological samples, and eliminating from the cohort biological samples that cannot be sequenced accurately or fail to align;
c. producing two or more different cells, tissues, organs, or organ types from each biological sample of the common cohort,
d. applying molecules or conditions to the biological samples to induce phenotypically distinct responses among the members of the cohort;
e. analyzing the responses by stimulus-response gene association studies; and
f. identifying a gene allele that is associated with a predetermined response to a stimulus when one cell, tissue, organ, or organ type of a donor is analyzed by a genetically diversified stimulus-response gene association study, but is not associated with the predetermined response to the stimulus when a different cell, tissue, organ, or organ type of the same donor is analyzed by a genetically diversified stimulus-response gene association study
wherein the cell, tissue, organ or organ type is affected by the identified gene allele.
